(19) United States
(12) Patent Application Publication
(10) Pub. No.: US 2021/0375464 A1
(43) Pub. Date: Dec. 2, 2021

(54) ASSISTANCE SYSTEM, ASSISTANCE METHOD, AND ASSISTANCE PROGRAM

(71) Applicant: TERUMO KABUSHIKI KAISHA, Tokyo (JP)

(72) Inventors: Yuuki SAKAGUCHI, Shizuoka (JP); Osamu NOMURA, Shizuoka (JP); Yasushi KINOSHITA, Shizuoka (JP); Yuusuke SEKINE, Kanagawa (JP)

(73) Assignee: TERUMO KABUSHIKI KAISHA, Tokyo (JP)

(21) Appl. No.: 17/400,606

(22) Filed: Aug. 12, 2021

Related U.S. Application Data
(63) Continuation of application No. 16/766,917, filed on May 26, 2020.

(30) Foreign Application Priority Data
Nov. 30, 2017 (JP) 2017-230847

(51) Int. Cl. G16H 50/20; G16H 40/20; G16H 10/60; G16H 80/00

(52) U.S. Cl. CPC G16H 50/20; G16H 80/00; G16H 10/60; G16H 40/20

(57) ABSTRACT

An assistance system, an assistance method, and an assistance program, which contribute to reduced medical expenses. An assistance system includes a data acquisition unit that acquires medical examination scheduled person data relating to a medical examination scheduled person having a scheduled medical examination in a medical institution, visit data relating to a visit history of the medical examination scheduled person who visits the medical institution, and medical examination data relating to a medical examination content of the medical examination scheduled person who received a medical examination in the medical institution in the past, a learning unit that performs machine learning by using the medical examination scheduled person data, the visit data, and the medical examination data, and a presentation unit that presents whether or not the medical examination is required for the medical examination scheduled person, based on a result of the machine learning.

### CROSS-REFERENCE TO RELATED APPLICATIONS

This application is a continuation of U.S. application Ser. No. 16/766,917, filed on May 26, 2020, which is a U.S. National Stage Application of PCT/JP2018/028728, filed on Jul. 31, 2018, which claims priority to Japanese Application No. 2017-230847, filed on Nov. 30, 2017, the entire contents of all three of which are incorporated herein by reference.

### FIELD OF THE DISCLOSURE

The present disclosure relates to an assistance system, an assistance method, and an assistance program, which assist a medical examination performed by a health care worker.

### BACKGROUND

In recent years, Japan has entered a super-aging society, and there is growing concern about insufficient health care workers and poor medical care quality. Therefore, in order to achieve an efficient medical care and to improve medical care quality, regional medical cooperation has been promoted in treating a patient in cooperation with a plurality of medical institutions.

For example, Pamphlet of International Publication No. 2014/097466 discloses a regional medical cooperation system that assists patient introduction between the medical institutions.

One of major problems in an aging society is rising medical expenses as social security expenses. There are various reasons for the rising medical expenses, such as expensive therapeutic drug launching and an advanced medical care. However, in particular, excessive medical examinations requested by elderly persons are regarded as one of the problems.

For example, the elderly persons are difficult to determine their own health conditions in a case where they are ill. Accordingly, the elderly persons actively visit medical institutions such as hospitals. In addition, some of the elderly persons, even though they are aware that a medical examination at the medical institution is actually unnecessary, in order to ease psychological loneliness, often visit the medical institution serving as a community where many elderly persons stay. As a result, there are problems such as increasing medical expenses, increasing workloads of health care worker such as doctors, and excessive medicine prescriptions.

On the other hand, while the health care worker recognizes that the elderly person does not need a prescription, there is a possibility that the health care worker may prescribe the medicines such as drugs for the elderly person who visits the hospital.

As described above, a healthy elderly person receives the medical examinations such as “first-of-all medical treatment”, “self-comfort medical treatment”, and “unnecessary medical treatment”. Accordingly, these behaviors lead to the “excessive medicine prescriptions”. As a result, there is a problem of the rising medical expenses.

### SUMMARY

The present disclosure is made in view of the above-described circumstances, and an object thereof is to provide an assistance system, an assistance method, and an assistance program, which contribute to reduced medical expenses.

According to the present disclosure, in order to achieve the above-described object, there is provided an assistance system for assisting a medical examination performed by a health care worker. The assistance system includes a data acquisition unit that acquires medical examination scheduled person data relating to a medical examination scheduled person having a scheduled medical examination in a medical institution, visit data relating to a visit history of the medical examination scheduled person who visits the medical institution, and medical examination data relating to a medical examination content of the medical examination scheduled person who received a medical examination in the medical institution in the past, a learning unit that performs machine learning by using the medical examination scheduled person data, the visit data, and the medical examination data, and a presentation unit that presents whether or not the medical examination is required for the medical examination scheduled person, based on a result of the machine learning.

According to the present disclosure, in order to achieve the above-described object, there is provided an assistance method for assisting a medical examination performed by a health care worker. The assistance method includes a data acquisition step of acquiring medical examination scheduled person data relating to a medical examination scheduled person having a scheduled medical examination in a medical institution, visit data relating to a visit history of the medical examination scheduled person who visits the medical institution, and medical examination data relating to a medical examination content of the medical examination scheduled person who received a medical examination in the medical institution in the past, a learning step of performing machine learning by using the medical examination scheduled person data, the visit data, and the medical examination data, and a presentation step of presenting whether or not the medical examination is required for the medical examination scheduled person, based on a result of the machine learning.

According to the present disclosure, in order to achieve the above-described object, there is provided an assistance program that causes a computer to execute a process for assisting a medical examination performed by a health care worker. The process includes a data acquisition step of acquiring medical examination scheduled person data relating to a medical examination scheduled person having a scheduled medical examination in a medical institution, visit data relating to a visit history of the medical examination scheduled person who visits the medical institution, and medical examination data relating to a medical examination content of the medical examination scheduled person who received a medical examination in the medical institution in the past, a learning step of performing machine learning by using the medical examination scheduled person data, the visit data, and the medical examination data, and a presentation step of presenting whether or not the medical examination is required for the medical examination scheduled person, based on a result of the machine learning.

According to the present disclosure, whether or not a medical examination performed by a health care worker is required for a medical examination scheduled person is presented, based on a result of machine learning. The health care worker can refer to a presented content to avoid the medical examination for the medical examination scheduled person who does not need the medical examination. As a result, it is possible to prevent increasing workloads of the health care worker and to prevent medicines from being excessively prescribed for elderly persons who visit a medical institution. Therefore, medical expenses can be effectively reduced.

In accordance with an aspect, a system is disclosed for assisting a health care worker in performing a medical examination, the system comprising: a processor configured to: receive a request from a patient for a medical examination at a medical institution; schedule the medical examination for the patient at the medical institution; acquire medical data relating to the medical examination scheduled by the patient at the medical institution, visit data relating to a visit history of the patient for one or more previous visits at the medical institution, and medical examination data relating to one or more medical examinations received at the medical institution by the patient; perform machine learning using the medical data, the visit data, and the medical examination data of the patient; and present whether or not the medical examination at the medical institution is required to the patient based on a result of the machine learning.

In accordance with another aspect, a method is disclosed for assisting a medical examination performed by a health care worker, the method comprising: receiving a request from a patient for a medical examination at a medical institution; scheduling the medical examination for the patient at the medical institution; acquiring medical examination scheduled person data relating to a medical examination scheduled person having a scheduled medical examination in a medical institution, visit data relating to a visit history of the medical examination scheduled person who visits the medical institution, and medical examination data relating to a medical examination content of the medical examination scheduled person who received a medical examination in the medical institution in the past; performing machine learning by using the medical examination scheduled person data, the visit data, and the medical examination data; and presenting whether or not the medical examination is required to the medical examination scheduled person, based on a result of the machine learning.

In accordance with an aspect, a non-transitory computer readable medium (CRM) storing computer program code executed by a computer processor that executes a process for assisting a medical examination performed by a health care worker is disclosed, the process comprising: receiving request from a patient for a medical examination at a medical institution; scheduling the medical examination for the patient at the medical institution; acquiring medical examination scheduled person data relating to a medical examination scheduled person having a scheduled medical examination in a medical institution, visit data relating to a visit history of the medical examination scheduled person who visits the medical institution, and medical examination data relating to a medical examination content of the medical examination scheduled person who received a medical examination in the medical institution in the past; performing machine learning by using the medical examination scheduled person data, the visit data, and the medical examination data; and presenting whether or not the medical examination is required to the medical examination scheduled person, based on a result of the machine learning.

### BRIEF DESCRIPTION OF DRAWINGS

FIG. 1 is a diagram illustrating an outline of an assistance system according to the present embodiment.

FIG. 2 is a diagram illustrating a state where the assistance system according to the present embodiment is connected to a medical institution terminal and a terminal of a medical examination scheduled person via a network.

FIG. 3A is a block diagram illustrating a hardware configuration of the assistance system according to the present embodiment.

FIG. 3B is a block diagram illustrating a functional configuration of the assistance system according to the present embodiment.

FIG. 4A is a view illustrating medical examination scheduled person data, visit data, and medical examination data of the assistance system according to the present embodiment.

FIG. 4B is a view illustrating prescription data of the assistance system according to the present embodiment.

FIG. 4C is a view illustrating regional data of the assistance system according to the present embodiment.

FIG. 4D is a view illustrating weather data of the assistance system according to the present embodiment.

FIG. 4E is a view illustrating medical institution data of the assistance system according to the present embodiment.

FIG. 5 is a flowchart illustrating an assistance method according to the present embodiment.

FIG. 6 is a view illustrating a presentation content and a presentation basis which are displayed on a display of the medical institution terminal.

### DETAILED DESCRIPTION

Hereinafter, an embodiment according to the present disclosure will be described with reference to the accompanying drawings. In the description of the drawings, the same reference numerals will be assigned to the same elements, and repeated description will be omitted. In addition, dimensional ratios in the drawings are exaggerated for convenience of the description, and may be different from actual ratios in some cases.

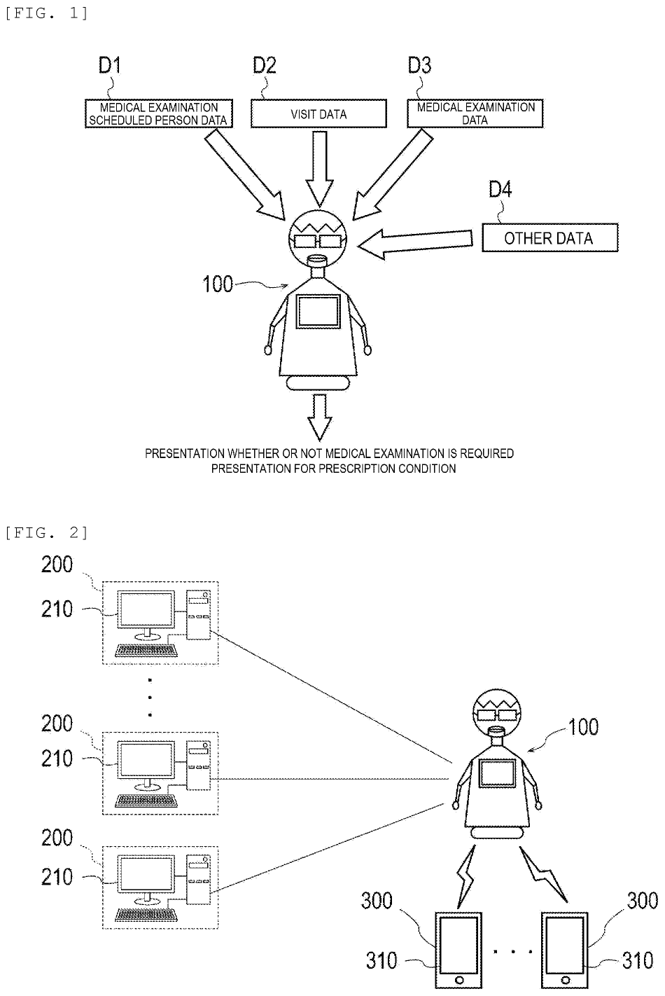

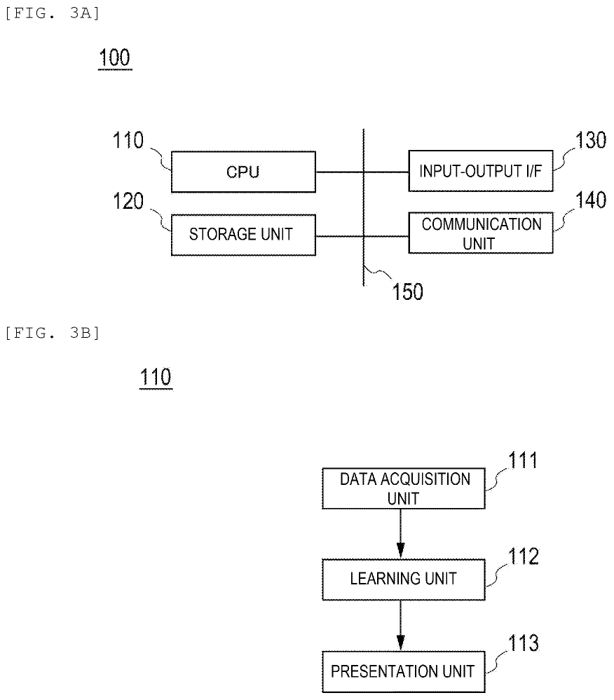

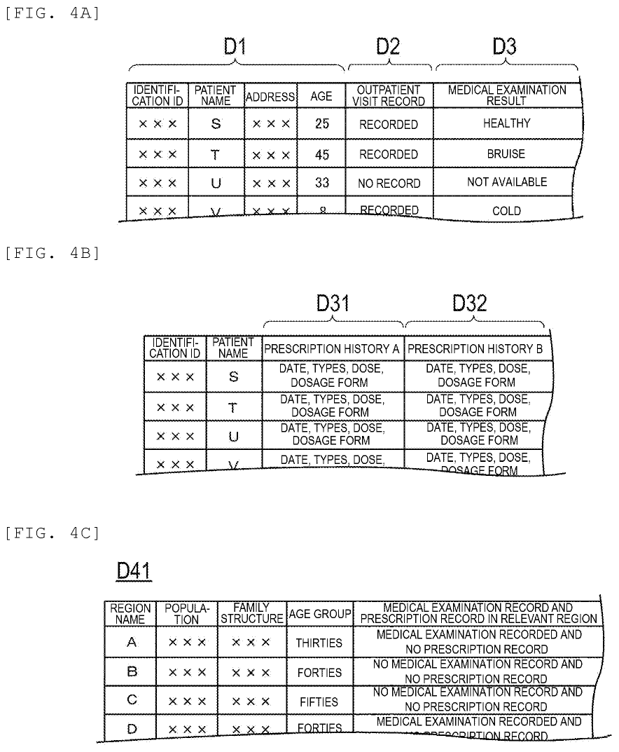

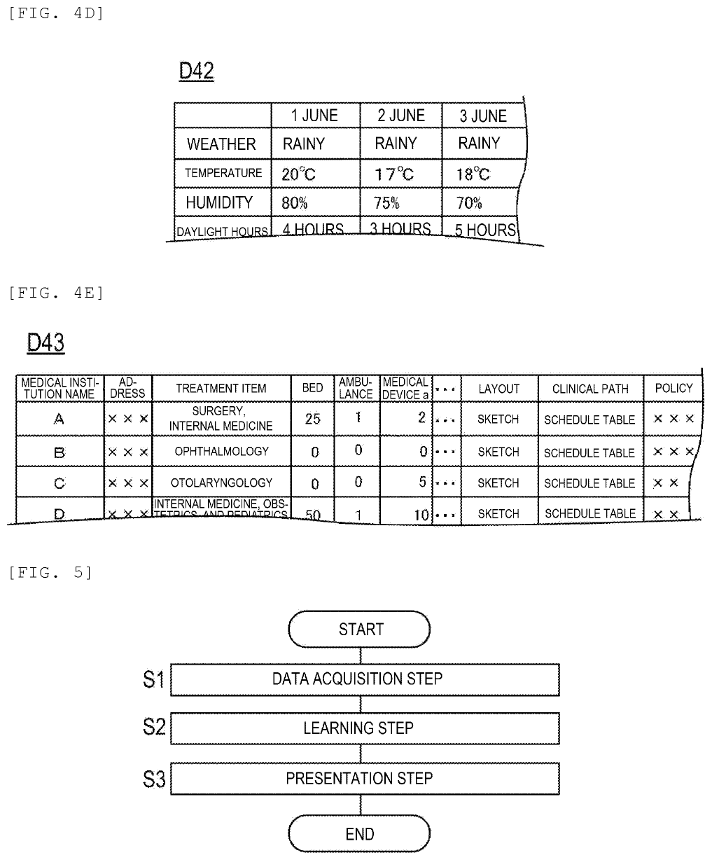

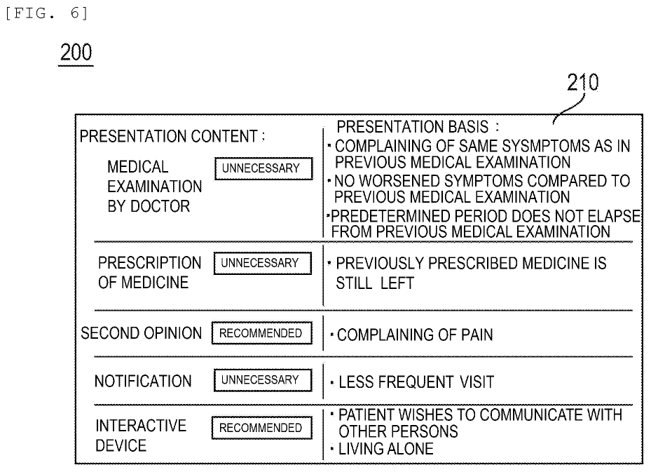

FIGS. 1 and 2 are diagrams for describing an overall configuration of an assistance system 100 according to the present embodiment. FIGS. 3A and 3B are diagrams for describing each unit of the assistance system 100. FIGS. 4A to 4E are views for describing data handled by the assistance system 100.

As illustrated in FIG. 1, the assistance system 100 is a system which uses medical examination scheduled person data D1, visit data D2, medical examination data D3, and other data D4 (regional data D41, weather data D42, and medical institution data D43) to present whether or not a medical examination is required for a medical examination scheduled person who wishes to receive the medical examination. Furthermore, the assistance system 100 presents prescription conditions of medicines (for example, whether or not a prescription of drugs is required, a type of the drugs, a dose of the drugs, and a dosage form of the drugs). Although not particularly limited, a “medical institution” means, for example, a facility where doctors and nurses provide medical cares for the medical examination scheduled person. For example, the medical institution includes hospitals and clinics. Although not particularly limited, a “specific (prescribed) region” means, for example, a region divided by a municipal unit, a prefecture unit, or a country unit.

As illustrated in FIG. 2, the assistance system 100 is connected to a medical institution terminal 200 of each medical institution and a medical examinee terminal 300 owned by each medical examination scheduled person via a network. The assistance system 100 is configured to function as a server that transmits and receives data between the medical institution terminal 200 and the medical examinee terminal 300. The medical examination scheduled person such as an elderly person can operate the medical examinee terminal 300 when visiting the medical institution or before visiting the medical institution. In this manner, the medical examination scheduled person can receive presentation of a medical examination policy from the assistance system 100. In addition, a health care worker (doctor or nurse) can confirm a medical examination policy through the medical institution terminal 200. For example, the network can adopt a wireless communication method using a communication function such as Wifi (registered trademark) or Bluetooth (registered trademark), other non-contact wireless communication, or wired communication.

In the present embodiment, the assistance system 100 is configured to include an interactive device capable of communicating with a person through a dialog. As the interactive device, for example, a robot equipped with an AI and having an interactive function can be used. For example, the interactive device can be equipped with a display capable of displaying a still image or a moving image, a speaker capable of outputting sound or music, and a camera function capable of capturing a still image or a moving image. Although not particularly limited, an exterior design of the interactive robot can include, for example, a humanoid type and an animal type.

Hereinafter, the assistance system 100 will be described in detail.

The hardware configuration of the assistance system 100 will be described.

Although not particularly limited, the assistance system 100 can be configured to include, for example, a mainframe or a computer cluster. As illustrated in FIG. 3A, the assistance system 100 includes a central processing unit (CPU) 110, a storage unit 120, an input-output I/F 130, and a communication unit 140. The CPU 110, the storage unit 120, the input-output I/F 130, and the communication unit 140 are connected to a bus 150, and transmit and receive data to and from each other via the bus 150.

The CPU 110 controls each unit, and performs various arithmetic processes in accordance with various programs stored in the storage unit 120.

The storage unit 120 is configured to include a read only memory (ROM) for storing various programs or various data items, a random access memory (RAM) for temporarily storing programs or data as a work region, and a hard disk for storing various programs including an operating system or various data items.

The input-output I/F 130 is an interface for connecting input devices such as a keyboard, a mouse, a scanner, and a microphone and output devices such as a display, a speaker, and a printer.

The communication unit 140 is an interface for communicating with the medical institution terminal 200 and the medical examinee terminal 300.

Next, a main function of the assistance system 100 will be described.

The storage unit 120 stores various data such as medical examination scheduled person data D1, visit data D2, medical examination data D3, and other data D4. In addition, the storage unit 120 stores an assistance program for providing an assistance method according to the present embodiment.

As illustrated in FIG. 3B, the CPU 110 functions as a data acquisition unit 111, a learning unit 112, and a presentation unit 113 by executing the assistance program stored in the storage unit 120.

The data acquisition unit 111 will be described.

The data acquisition unit 111 acquires the medical examination scheduled person data D1, the visit data D2, the medical examination data D3, and other data D4.

As illustrated in FIG. 4A, for example, the medical examination scheduled person data D1 includes an identification ID of the medical examination scheduled person (for example, data that can be acquired from an individual number), and a name, an address, and an age of the medical examination scheduled person. For example, the visit data D2 includes a visit record (records of visits to the medical institution). For example, the medical examination data D3 includes a result of the medical examination when the medical examination scheduled person visited the medical institution last time and a result of the medical examination when the medical examination scheduled person visited the medical institution before the previous visit. The medical examination data D3 can also include data acquired during the medical examination (during an outcall) in a case where the medical examination scheduled person has an experience in home medical care or home nursing.

For example, the medical examination scheduled person data D1 can include data relating to genetic information of the medical examination scheduled person. The genetic information may include not only genetic information on the medical examination scheduled person but also genetic information of relatives. For example, the genetic information can be configured to include a DNA test result. For example, when a disease of the medical examination scheduled person is determined, the genetic information can be used to determine whether the disease is strongly affected by genetic factors.

The medical examination scheduled person data D1, the visit data D2, and the medical examination data D3 are stored in the storage unit 120 in a state of being associated with each medical examination scheduled person. In addition, each of the data D1, D2, and D3 can be stored and managed using a known electronic medical record, for example.

As illustrated in FIG. 4B, the medical examination data D3 can include medical institution prescription data (prescription data) D31 and pharmacy prescription data (prescription data) D32. For example, the medical institution prescription data D31 includes various data relating to a prescription in a case where the medicine (for example, a drug) was prescribed for the medical examination scheduled person in the medical institution in the past. For example, the medical institution prescription data D31 includes data relating to a date and a time of the prescription, or a type, a prescription dose, and a dosage form of the medicine. The pharmacy prescription data D32 includes data relating to the medicine actually prescribed for the medical examination scheduled person by a pharmacy, based on the prescription provided by the medical institution. For example, as in the medical institution prescription data D31, the pharmacy prescription data D32 includes data relating to the date and the time of the prescription, or the type, the prescription dose, and the dosage form of the medicine (prescription history written on a medicine notebook). The medicine according to the present embodiment includes a so-called digital medicine equipped with a digital function (for example, a function to acquire biological information by detecting the biological information of a biological organ after the medicine is taken). For example, information relating to the medical examination scheduled person, which is acquired by the digital medicine, can be shared among the medical institution, the medical examination scheduled person, and the health care worker, or can be used in monitoring a medicine taking state of the medical examination scheduled person.

For example, the data acquisition unit 111 acquires the medical examination scheduled person data D1, the visit data D2, and the medical examination data D3 from the medical institution terminal 200 of each medical institution and the medical examinee terminal 300 of each medical examination scheduled person.

The other data D4 which is an acquisition target of the data acquisition unit 111 can include the regional data D41 illustrated in FIG. 4C, the weather data D42 illustrated in FIG. 4D, and the medical institution data D43 illustrated in FIG. 4E.

As illustrated in FIG. 4C, the regional data D41 includes a name of a specific region, population in the specific region, major family structures in the specific region (for example, an average value of the number of family members in the specific region), age groups in the specific region (for example, an average value of age groups in the specific region), and information on whether the medical examination scheduled person has records of medical examination or the prescription in the specific region. For example, the regional data D41 can include data on diseases which are endemic in the specific region. In addition, for example, the regional data D41 can include data relating to traffic information in the specific region. For example, the data relating to the traffic information includes a distance from a home of the medical examination scheduled person to the medical institution, and a type of available transportation systems (for example, buses or trains).

As illustrated in FIG. 4D, the weather data D42 includes data relating to weather (meteorology) in the surrounding environment of each medical institution. The weather data D42 includes the weather, the temperature, the humidity, and daylight hours of the surrounding environment.

For example, the data acquisition unit 111 can acquire the regional data D41 and the weather data D42 from the Internet.

As illustrated in FIG. 4E, the medical institution data D43 includes data relating to names (medical institution name), addresses, medical specialities, a number of facilities held (devices including beds, ambulances, medical devices, and business machines), layouts, clinical paths, policies, and doctors of each medical institution. The data is stored in the storage unit 120 in a state of being associated with each medical institution. For example, the layout data can be configured to include a medical institution sketch indicating positions and distances of the respective facilities, medical examination rooms, test rooms, surgery rooms, a nurse station, a general ward, an intensive care unit (ICU), and a high care unit (HCU). For example, the clinical path data can be configured to include a schedule table that summarizes a schedule from hospital admission to hospital discharge of a plurality of the medical examination scheduled persons. For example, the policy data includes data relating to education policies for training and the like, and data relating to medical policies for priority medical cares and the like. In addition, although not illustrated in the drawing, for example, the doctor data includes data relating to doctor names, medical specialities, treatment experiences, surgery experiences, and work schedules. The data is stored in the storage unit 120 in a state of being associated with each doctor.

In addition, for example, the medical institution data D43 can include data relating to a congestion status of the medical institution. For example, the data relating to the congestion status includes a congestion status (outpatient congestion status or hospital admission congestion status) of the medical institution located within a prescribed range from a home of the medical examination scheduled person. For example, when the medical examination scheduled person visits a prescribed medical institution, the assistance system 100 can provide information (timetables or transit guidance) on the most suitable transportation system for the medical examination scheduled person, based on data relating to the traffic information or data relating to the congestion status, can recommend a doctor having excellent therapeutic outcomes for a specific disease, or can present the medical institution for which the doctor works. In addition, the assistance system 100 may automatically present the medical institution with the transportation system, and may automatically reserve the medical examination in accordance with an arrival time at the medical institution.

In addition, for example, the other data D4 can include reuse data relating to the medical devices and the medicines. For example, the reuse data includes information relating to whether the medical devices can be reused by performing cleaning or sterilization. For example, as the above-described medical devices, single-use medical devices may be used, but medical devices (some configuration components of the medical devices) other than the single-use medical devices also may be used. In addition, for example, the reuse data can include information relating to surplus medicines. The information relating to the surplus medicines includes information relating to whether a drug (for example, a liquid drug) stored in a predetermined amount in a container such as a bottle can be used for the plurality of medical examination scheduled persons. For example, the drug can be treated as a reusable drug in a case where the drug stored in a specific container can be administered to the medical examination scheduled person and the drug stored in a similar container can be administered to another medical examination scheduled person.

For example, the reuse data can be acquired on a real-time basis from a hospital information system of the medical institution that owns the medical devices and the medicine which are reuse targets.

For example, the data acquisition unit 111 can acquire medical data as other information useful for assisting the health care worker. For example, the medical data includes data relating to medical knowledge, which includes disease data relating to diseases (disease name, symptoms, and whether receiving the treatment is required), treatment data relating to treatment (treatment method, time required for the treatment, required facilities and drugs, and wholesale prices thereof), and data relating to a medical insurance system. For example, the data acquisition unit 111 can acquire the medical data from the Internet, or can acquire the medical data from electronic data of medical specialty books captured by a scanner or the like.

Next, the learning unit 112 will be described.

The learning unit 112 performs machine learning by using the medical examination scheduled person data D1, the visit data D2, the medical examination data D3, and other data D4. In the description herein, the “machine learning” means analyzing input data by using an algorithm, extracting useful rules and criteria from an analysis result thereof, and developing the algorithm.

The assistance system 100 according to the present embodiment presents whether or not the medical examination performed by the health care worker is required, and also presents prescription conditions of the medicines. Based on each data described above, the assistance system 100 performs the machine learning so that the presentation contents do not become invalid. The learning unit 112 performs the machine learning. In this manner, the assistance system 100 predicts current and future dynamic states of the medical examination scheduled person from past dynamic states of the medical examination scheduled person (frequency of visits to the medical institution, contents of the medical examination, results of the medical examination, prescriptions of the medicines, and usages of the medicines). The assistance system 100 proposes suitable countermeasures to the health care worker, based on a prediction result. For example, the learning unit 112 can learn the prescription condition of preferable medicines, based on the medical institution prescription data D31 and/or the pharmacy prescription data D32 acquired from a plurality of persons.

Specifically, in a case where the medical examination scheduled person who visits the medical institution or the medical examination scheduled person before visiting the medical institution requests for the medical examination, the presentation unit 113 presents whether or not the medical examination is required to the health care worker, based on a result of the machine learning of the learning unit 112. In addition, the presentation unit 113 also presents the prescription conditions of the medicines prescribed by the health care worker for the medical examination scheduled person. Here, for example, the prescription condition includes determining whether or not the prescription of the medicine is required, and specifying the type, the prescription dose, the usage, the dosage form of the drugs. In addition, as an example of the presentation performed by the presentation unit 113, for example, based on the medical institution prescription data D31 and/or the pharmacy prescription data D32 acquired from a plurality of persons, the presentation unit 113 may present sharing the surplus medicine within one household (for example, a married couple or parent and child). The presentation unit 113 may present using the medicine of someone who no longer needs to take the medicine for some reason for another person in a predetermined population. Alternatively, the presentation unit 113 may present the persons having the prescription of the same medicine to jointly purchase the medicine so as to reduce the purchasing costs.

When presenting whether or not the medical examination performed by the health care worker is required and the prescription conditions of the medicines, the presentation unit 113 presents the presentation contents and the presentation basis that leads to the presentation. For example, in the present embodiment, as will be described later, in a case where it is determined that the medical examination performed by the health care worker is not required, the basis is presented, based on each data. In a case where a plurality of bases are presented, the plurality of bases can be presented. The health care worker can satisfactorily adopt the respective presentation contents by being presented whether or not the medical examination performed by the health care worker is required and the prescription conditions of the medicines together with the basis. As a method of presenting the basis, for example, a relationship between data items may be displayed using a graph or a table, or an event serving as a factor of the basis may be specifically displayed together with a numerical value such as a contribution ratio.

In the present embodiment, the presentation unit 113 performs the presentation in a case where the health care worker or the medical examination scheduled person requests for the presentation. However, timing for the presentation by the presentation unit 113 is not particularly limited. For example, the presentation unit 113 may automatically acquire the data on an irregular or regular basis. Even if the health care worker or the medical examination scheduled person does not request for the presentation, in a case where it is predicted that the medical examination scheduled person visits the medical institution, the presentation unit 113 may automatically present a suitable countermeasure policy for the medical examination scheduled person to the medical institution, or the health care worker. In addition, for example, the presentation unit 113 may acquire the data relating to the dynamic state of the medical examination scheduled person on an irregular or regular basis, and may present future predictions of the treatment policy to the medical examination scheduled person who is predicted to visit the medical institution.

FIGS. 5 and 6 are figures for describing the assistance method according to the present embodiment. Hereinafter, the assistance method according to the present embodiment will be described with reference to FIGS. 5 and 6.

Referring to FIG. 5, the assistance method schematically includes a data acquisition step (S1) of acquiring the medical examination scheduled person data D1, the visit data D2, the medical examination data D3, and other data D4, a learning step (S2) of performing mechanical learning by using the medical examination scheduled person data D1, the visit data D2, the medical examination data D3, and other data D4, and a presentation step (S3) of presenting whether or not the medical examination performed by the health care worker is required and the prescription conditions of the medicines, based on a result of the machine learning. Hereinafter, each step will be described.

The algorithm of the machine learning is generally classified into supervised learning, unsupervised learning, and reinforcement learning. In the algorithm of the supervised learning, a set of input data and result data is provided for the learning unit 112 to perform the machine learning. In the algorithm of the unsupervised learning, only the input data is provided in a large amount for the learning unit 112 to perform the machine learning. In the algorithm of the reinforcement learning, an environment is changed, based on a solution output by the algorithm, and a correction is added, based on a reward indicating how correct the output solution is. The algorithm of the machine learning of the learning unit 112 may be any one of the supervised learning, the unsupervised learning, and the reinforcement learning. In the present embodiment, a case where the learning unit 112 performs the machine learning by using the algorithm of the supervised learning will be described as an example.

First, the data acquisition step (S1) will be described.

In the data acquisition step (S1), the data acquisition unit 111 acquires the medical examination scheduled person data D1, the visit data D2, the medical examination data D3, and other data D4, and stores the data in the storage unit 120. The timing for the data acquisition unit 111 to acquire the medical examination scheduled person data D1, the visit data D2, the medical examination data D3, and other data D4 is not particularly limited. For example, the data may be acquired every predetermined time, or may be acquired at the timing when the data is changed. The data acquisition unit 111 acquires the medical examination scheduled person data D1, the visit data D2, the medical examination data D3, and other data D4 over a predetermined period, and stores the data in the storage unit 120. Therefore, a large amount of the input data and the solution data for performing the supervised learning are stored in the storage unit 120.

For example, in the present embodiment, when the medical examination scheduled person visits the medical institution, the medical institution acquires and confirms each data of the medical examination scheduled person (the medical examination scheduled person data D1, the visit data D2, and the medical examination data D3) inside or outside a predetermined region, based on a medical examination voucher, a health insurance card, shared data of the regional medical care using electronic medical records, and an individual number. In addition, at this time, countermeasures to the medical examination scheduled person are dealt with by one or more interactive devices, and hearing of a testimony relating to the medical examination is received from the medical examination scheduled person. A result of the hearing is used together with each data in the learning step (to be described later).

A method of acquiring information from the medical examination scheduled person is not limited to a method of acquiring linguistic information through the hearing as described above. For example, the assistance system 100 may acquire biological information. For example, the method of acquiring the biological information includes a method of acquiring a body temperature or oxygen saturation by using infrared rays, and a method of acquiring a degree of progression of arteriosclerosis by measuring pulse waves of peripheral blood vessels. In addition, the assistance system 100 may acquire information relating to a reaction of the medical examination scheduled person during the hearing (the degree of facial redness tide or motor function) via the interactive device. In addition, the assistance system 100 can be provided with the algorithm that determines behavior authenticity of the medical examination scheduled person, based on the information obtained using the hearing and the above-described respective methods, and that confirms validity of each of the information obtained from the medical examination scheduled person.

The information can be acquired from the medical examination scheduled person only by the interactive device included in the assistance system 100. However, for example, the information may be acquired by a person (health care worker), or may be acquired by both the interactive device and the person. For example, regarding an item of which information processing is not smoothly performed using the interactive device alone, the person communicates with the medical examination scheduled person through the interactive device, and inputs the acquired information. In this manner, the information can be more accurately and smoothly acquired from the medical examination scheduled person.

Next, the learning step (S2) will be described.

In the learning step (S2), the learning unit 112 applies the algorithm of the supervised learning to a large data set stored in the storage unit 120. The algorithm of the supervised learning is not particularly limited. However, for example, known algorithms such as a least squares method, a linear regression, an autoregression, and a neural network can be used.

Based on the acquired data, the learning unit 112 predicts current and future dynamic states relating to the visit of the medical examination scheduled person to the medical institution. In addition, referring to the above-described hearing result and predicted result, presentation of whether or not the medical examination by the health care worker is required, and that of the prescription dynamic states of the medicines are performed.

In addition, with regard to the medical device used for the surgery or the treatment, the learning unit 112 can perform the machine learning on information useful for determining the reuse of the medical device, based on the information regarding whether or not the medical device can be reused, which method (cleaning or sterilization method) enables the medical device to be reused in a case where the medical device can be reused, and which configuration member of the medical device can be reused. In addition, with regard to the medicine used for the surgery or the medical treatment, the learning unit 113 can perform the machine learning on the information useful for determining the reuse of the medicine, based on the information regarding whether or not the medicine can be reused and which method (storage method of the medicine or method of providing the medicine for the medical examination scheduled person) enables the medicine to be reused in a case where the medicine can be reused. The presentation unit 113 can provide the medical institution with the information relating to the reuse of the medical device or the medicine by presenting a learning result of the above-described machine learning. The medical institution can effectively reduce medical expenses in such a way that the learning result relating to the reuse is acquired from or shared with one specific medical institution or a plurality of the medical institutions.

Next, the presentation step (S3) will be described.

For example, as illustrated in FIG. 6, the presentation unit 113 can cause the display 210 of the medical institution terminal 200 to display the presentation content and the presentation basis. For example, the presentation content and the presentation basis can be displayed on a display 310 (refer to FIG. 1) of the medical examinee terminal 300 owned by the medical examination scheduled person or a display included in the interactive device.

An example of the presentation content and the presentation basis will be described with reference to FIG. 6.

For example, in a case where it is determined as the presentation content that the medical examination performed by the doctor is not required, a main reason leading to the determination result is displayed as the presentation basis. In addition, with regard to whether or not the medical examination is required and the prescription conditions of the medicines, the determination result is displayed as the presentation content.

As illustrated in FIG. 6, the presentation content includes a second opinion, for example. For example, the second opinion includes both the determination on whether or not the medical examination performed by the health care worker is required and the determination on the prescription conditions of the medicines. In addition, if it is determined that a new prescription of the medicines is required based on the second opinion (in a case where a drug different from that of the previous prescription is prescribed), the recommendation of the new prescription is presented. In a case where a medicine the same as that of the previous prescription is prescribed, the remaining amount of the medicine is predicted based on each of the prescription data D31 and D32 (refer to FIG. 4B), and the recommendation of the prescription to make up for a shortfall is presented.

As illustrated in FIG. 6, for example, the presentation content includes a notification. In a case where it is determined based on a result of the hearing of the medical examination scheduled person that the previous medical examination or the previous prescription of the medicine is not proper, the notification proposes that the determination result to the medical examination scheduled person, the medical institution, and relatives of the medical examination scheduled person. The presentation unit 113 presents to notify a public institution of the determination result, for example, in a case where the determination result is obtained indicating that the medical examination scheduled person intentionally wishes to receive a repeated examination or the prescription of the medicine is intentionally duplicated.

In addition, as illustrated in FIG. 6, for example, the presentation content includes a usage of the interactive device. If it is determined that the medical examination scheduled person does not visit the medical institution to receive the medical examination, a conversation (communication) is made using the interactive device. In this manner, the medical examination scheduled person can be satisfied even if the medical examination is not performed by the health care worker. Therefore, the medical examination scheduled person can be smoothly recommended to return home.

For example, in a case where the presentation unit 113 presents that the medical examination performed by the health care worker is not required, the presentation unit 113 may present a method other than the conversation using the interactive device, as another medical examination practice that replaces the medical examination by the health care worker. For example, the presentation unit 113 can present a conversation with a volunteer staff, a conversation with another medical examination scheduled person, or a friendship with an animal.

In a case where a countermeasure to a specific medical examination scheduled person is presented, the assistance system 100 may cause the data acquisition unit 111 to acquire again the data such as the medical examination scheduled person data D1, the visit data D2, and the medical examination data D3. Then, the learning unit 112 may perform the machine learning again by using newly acquired data, and may update a learning model. Based on the updated learning model, for example, the assistance system 100 can predict the future dynamic states of the same medical examination scheduled person or a different medical examination scheduled person, can accumulate the result as new data, and can use the result for the next proposal.

As described above, the assistance system 100 according to the present embodiment includes the data acquisition unit 111 that acquires the medical examination scheduled person data D1 relating to the medical examination scheduled person having the scheduled medical examination in the medical institution, the visit data D2 relating to the visit history of the medical examination scheduled person who visits the medical institution, and the medical examination data D3 relating to the medical examination content in which the medical examination scheduled person received the medical examination in the medical institution in the past, the learning unit 112 that performs the machine learning by using the medical examination scheduled person data D1, the visit data D2, and the medical examination data D3, and the presentation unit 113 that presents whether or not the medical examination is required for the medical examination scheduled person, based on the result of the machine learning.

As described above, based on the result of the machine learning, the assistance system 100 presents whether or not the medical examination performed by the health care worker is required for the medical examination scheduled person. The health care worker can refer to the presented content to avoid the medical examination for the medical examination scheduled person who does not need the medical examination. As a result, it is possible to prevent increasing workloads of the health care worker and to prevent the medicines from being excessively prescribed for elderly persons who visit the medical institution. Therefore, medical expenses can be effectively reduced.

In addition, in a case where the presentation unit 113 presents that the medical examination is not required, the presentation unit 113 presents another medical examination practice that replaces the medical examination of the health care worker. Therefore, the medical examination scheduled person can be highly satisfied with visiting the medical institution, even in a case where the medical examination is not performed by the health care worker.

In addition, the presentation unit 113 presents the communication with the medical examination scheduled person by using the interactive device, as another medical examination practice. Therefore, the medical examination scheduled person can be further satisfied while the increase in the workloads of the health care worker is suppressed.

In addition, the medical examination data D3 includes prescription data D31 and D32 relating to the medicine prescribed for the medical examination scheduled person. The learning unit 112 learns the recommended prescription conditions of the medicines, based on the medical examination scheduled person data D1, the visit data D2, the medical examination data D3, and the prescription data D31 and D32. Then, the presentation unit 113 presents the prescription conditions of the medicines, based on the result of the machine learning. Therefore, the assistance system 100 can more properly determine whether or not the medicine needs to be prescribed. In a case where the medicine is prescribed, the assistance system 100 can provide a proper prescription dose and a proper type of the medicine.

In addition, the presentation unit 113 presents the presentation basis together with the presentation content. Therefore, the health care worker or the medical examination scheduled person can satisfactorily adopt the presented content.

In addition, the assistance method according to the present embodiment includes the data acquisition step (S1) of acquiring the medical examination scheduled person data D1 relating to the medical examination scheduled person having the scheduled medical examination in the medical institution, the visit data D2 relating to the visit history of the medical examination scheduled person who visits the medical institution, and the medical examination data D3 relating to the medical examination content in which the medical examination scheduled person received the medical examination in the medical institution in the past, the learning step (S2) of performing the machine learning by using the medical examination scheduled person data D1, the visit data D2, and the medical examination data D3, and the presentation step (S3) of presenting whether or not the medical examination is required for the medical examination scheduled person, based on the result of the machine learning. Therefore, the health care worker can refer to the presented content to avoid the medical examination for the medical examination scheduled person who does not need the medical examination. As a result, it is possible to prevent increasing workloads of the health care worker and to prevent the medicines from being excessively prescribed for elderly persons who visit the medical institution. Therefore, medical expenses can be effectively reduced.

In addition, the assistance program according to the present embodiment causes a computer to execute a process including the data acquisition step (S1) of acquiring the medical examination scheduled person data D1 relating to the medical examination scheduled person having the scheduled medical examination in the medical institution, the visit data D2 relating to the visit history of the medical examination scheduled person who visits the medical institution, and the medical examination data D3 relating to the medical examination content in which the medical examination scheduled person received the medical examination in the medical institution in the past, the learning step (S2) of performing the machine learning by using the medical examination scheduled person data D1, the visit data D2, and the medical examination data D3, and the presentation step (S3) of presenting whether or not the medical examination is required for the medical examination scheduled person, based on the result of the machine learning. Therefore, the health care worker can refer to the presented content to avoid the medical examination for the medical examination scheduled person who does not need the medical examination. As a result, it is possible to prevent increasing workloads of the health care worker and to prevent the medicines from being excessively prescribed for elderly persons who visit the medical institution. Therefore, medical expenses can be effectively reduced.

Hitherto, the assistance system, the assistance method, and the assistance program according to the present disclosure have been described with reference to the embodiment. However, the present disclosure is not limited only to each configuration described herein, and can be appropriately modified based on the description in the appended claims.

For example, the assistance system, the assistance method, and the assistance program according to the above-described embodiment may share the acquired data and presentation content with a plurality of medical institutions, or may be used only for a single medical institution.

In addition, the data used for the machine learning by the assistance system according to the present disclosure is not particularly limited as long as at least the medical examination scheduled person data, the visit data, and the medical examination data are used. In addition, the presentation content is sufficient when including at least whether or not the medical examination is required for the medical examination scheduled person.

In addition, in a case where the medical examination data includes the prescription data, the prescription data is sufficient when including at least one of the medical institution prescription data and the pharmacy prescription data.

In addition, in the assistance system according to the above-described embodiment, the learning unit performs the machine learning by using the algorithm of the supervised learning. However, the algorithm used for the machine learning by the learning unit may be the algorithm of the unsupervised learning, or may be the algorithm of the reinforcement learning. In addition, the learning unit may perform the machine learning by using a plurality of types of the algorithms.

In addition, the means and the method for performing various processes in the assistance system according to the above-described embodiment may be realized by a dedicated hardware circuit or a programmed computer. In addition, for example, the assistance program may be provided by a computer-readable recording medium such as a compact disc read only memory (CD-ROM), or may be provided online via a network such as the Internet. In this case, the program recorded in the computer-readable recording medium is usually transferred to and stored in the storage unit such as a hard disk. In addition, the assistance program may be provided as single application software.

The detailed description above describes embodiments of an assistance system, an assistance method, and an assistance program, which assist a medical examination performed by a health care worker representing examples of the inventive system, method, and program disclosed here. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A system for assisting a health care worker in performing a medical examination, the system comprising:
a processor configured to:
receive a request from a patient for a medical examination at a medical institution;
schedule the medical examination for the patient at the medical institution;
acquire medical data relating to the medical examination scheduled by the patient at the medical institution, visit data relating to a visit history of the patient for one or more previous visits at the medical institution, and medical examination data relating to one or more medical examinations received at the medical institution by the patient;
perform machine learning using the medical data, the visit data, and the medical examination data of the patient; and
present whether or not the medical examination at the medical institution is required to the patient based on a result of the machine learning.

2. The assistance system according to claim 1, wherein in a case where the medical examination is not required, the processor is configured to present another medical examination practice that replaces the medical examination by the health care worker.

3. The assistance system according to claim 2, wherein as the other medical examination practice, the processor is configured to present communication with the medical examination scheduled person through an interactive device.

4. The assistance system according to claim 1, wherein the medical examination data includes prescription data relating to a medicine prescribed for the medical examination scheduled person, and the processor is configured to:
perform the machine learning on a recommended prescription condition of the medicine, based on the medical examination scheduled person data, the visit data, the medical examination data, and the prescription data, and
present the prescription condition, based on a result of the machine learning.

5. The assistance system according to claim 1, wherein the processor is configured to present a presentation basis together with a presentation content.

6. The assistance system according to claim 1, wherein the processor is further configured to:
automatically acquire the visit data and the medical examination data for the patient before the patient has requested another medical examination at the medical institution;
perform machine learning using the visit data and the medical examination data of the patient; and
automatically present a treatment policy for patient before the another medical examination at the medical institution has been requested based on a result of the machine learning.

7. The assistance system according to claim 1, wherein the visit data includes records of visits to the medical institution, and the medical examination data includes at least a result of the medical examination when the medical examination scheduled person visited the medical institution a last time and a result of the medical examination when the medical examination scheduled person visited the medical institution before the last time.

8. The assistance system according to claim 7, wherein the medical examination data includes data acquired during a medical examination of the patient from in home medical care or home nursing.

9. The assistance system according to claim 1, wherein the medical data includes data relating to genetic information of the patient, the genetic information including genetic information on the patient and genetic information of relatives of the patient.

10. The assistance system according to claim 1, wherein the processor is configured to:
present to the patient that the medical examination at the medical institution is not required; and
present to the patient that another medical examination at the medical institution should be scheduled with another health care worker.

11. A method for assisting a medical examination performed by a health care worker, the method comprising:
receiving a request from a patient for a medical examination at a medical institution;
scheduling the medical examination for the patient at the medical institution;
acquiring medical examination scheduled person data relating to a medical examination scheduled person having a scheduled medical examination in a medical institution, visit data relating to a visit history of the medical examination scheduled person who visits the medical institution, and medical examination data relating to a medical examination content of the medical examination scheduled person who received a medical examination in the medical institution in the past;
performing machine learning by using the medical examination scheduled person data, the visit data, and the medical examination data; and
presenting whether or not the medical examination is required to the medical examination scheduled person, based on a result of the machine learning.

12. The method according to claim 11, wherein in a case where the medical examination is not required, the processor is configured to present another medical examination practice that replaces the medical examination by the health care worker.

13. The method according to claim 12, wherein as the other medical examination practice, the processor is configured to present communication with the medical examination scheduled person through an interactive device.

14. The method according to claim 11, wherein the medical examination data includes prescription data relating to a medicine prescribed for the medical examination scheduled person, and the method further comprises:
performing the machine learning on a recommended prescription condition of the medicine, based on the medical examination scheduled person data, the visit data, the medical examination data, and the prescription data, and
presenting the prescription condition, based on a result of the machine learning.

15. The method according to claim 11, further comprising:
presenting a presentation basis together with a presentation content.

16. The method according to claim 11, further comprising:
automatically acquiring the visit data and the medical examination data for the patient before the patient has requested another medical examination at the medical institution;
performing machine learning using the visit data and the medical examination data of the patient; and
automatically presenting a treatment policy for patient before the another medical examination at the medical institution has been requested based on a result of the machine learning.

17. The method according to claim 11, wherein the visit data includes records of visits to the medical institution, and the medical examination data includes at least a result of the medical examination when the medical examination scheduled person visited the medical institution a last time and a result of the medical examination when the medical examination scheduled person visited the medical institution before the last time, and the medical examination data includes data acquired during a medical examination of the patient from in home medical care or home nursing.

18. The method according to claim 11, wherein the medical data includes data relating to genetic information of the patient, the genetic information including genetic information on the patient and genetic information of relatives of the patient.

19. The method according to claim 11, further comprising:
presenting to the patient that the medical examination at the medical institution is not required; and
presenting to the patient that another medical examination at the medical institution should be scheduled with a different health care worker.

20. A non-transitory computer readable medium (CRM) storing computer program code executed by a computer processor that executes a process for assisting a medical examination performed by a health care worker, the process comprising:
receiving request from a patient for a medical examination at a medical institution;
scheduling the medical examination for the patient at the medical institution;
acquiring medical examination scheduled person data relating to a medical examination scheduled person having a scheduled medical examination in a medical institution, visit data relating to a visit history of the medical examination scheduled person who visits the medical institution, and medical examination data relating to a medical examination content of the medical examination scheduled person who received a medical examination in the medical institution in the past;
performing machine learning by using the medical examination scheduled person data, the visit data, and the medical examination data; and
presenting whether or not the medical examination is required to the medical examination scheduled person, based on a result of the machine learning.
